(19) United States
(12) Patent Application Publication
(10) Pub. No.: US 2025/0281234 A1
(43) Pub. Date: Sep. 11, 2025

(54) DEVICES, SYSTEMS AND METHODS INCLUDING A VARIABLY INSULATED ELECTROSURGICAL GUIDEWIRE

(71) Applicant: ElectroWire Corporation, Lexington, SC (US)

(72) Inventors: Robert Leonardi, Lexington, SC (US); Peter J. D'Aquanni, Murrieta, CA (US)

(21) Appl. No.: 19/076,130

(22) Filed: Mar. 11, 2025

Related U.S. Application Data
(60) Provisional application No. 63/563,517, filed on Mar. 11, 2024.
(60) Provisional application No. 63/569,304, filed on Mar. 25, 2024.

(51) Int. Cl. A61B 18/14; A61B 18/00; A61B 18/12

(52) U.S. Cl. CPC A61B 18/1492; A61B 18/1206; A61B 2018/00083; A61B 2018/00136; A61B 2018/00142; A61B 2018/00702; A61B 2018/144

(57) ABSTRACT

An electrosurgical guidewire including a core wire including a distal core wire portion, a shaft portion, an active electrode, a first electrical insulation material, and a second electrical insulation material. The distal core wire portion is located at a distal portion of the electrosurgical guidewire. The shaft portion is located at a proximal portion of the electrosurgical guidewire and includes an electrical connection portion configured to provide an electrical connection to an electrosurgical generator. The first electrical insulation material has a first thickness covering the distal core wire portion. The second electrical insulation material has a second thickness covering the shaft portion. The first thickness is greater than the second thickness.

### CROSS REFERENCE TO RELATED APPLICATIONS

This application claims the benefit of U.S. Provisional Patent Application Ser. No. 63/569,304 filed Mar. 25, 2024 (pending), and U.S. Provisional Patent Application Ser. No. 63/563,517, filed Mar. 11, 2024 (pending), the disclosures of which are incorporated by reference herein in their entirety.

### TECHNICAL FIELD

The present disclosure generally relates to electrosurgical guidewires and, more particularly, to devices, systems and methods involving the use of electrically insulated electrosurgical guidewires.

### BACKGROUND

Transcatheter electrosurgery involves the use of an energized electrosurgical guidewire to vaporize tissue. The core wire of the electrosurgical guidewire is usually metallic (such as stainless steel) and runs the entire length of the guidewire. The distal tip or terminus of the electrosurgical guidewire usually serves as the “active electrode” in the system, where electric current density is highly concentrated to effect tissue vaporization. The remainder of the electrosurgical guidewire must be insulated to minimize charge dispersion and related loss of electrosurgical effect. The core wire outer diameter is the main determinant of mechanical guidewire performance (e.g. mechanical characteristics such as strength and flexibility), and the insulation is the main determinant of electrosurgical guidewire performance.

Electrosurgical guidewire insulation has been accomplished by uniformly covering the length of the guidewire with an electrical insulation material, such as spray coating or shrink tubing. In applications where outer guidewire diameter is constrained but a significant thickness (greater than 0.001 inches) of guidewire insulation is needed, core wire diameter may be reduced to allow an adequate thickness of insulation material. Thus, mechanical performance of the electrosurgical guidewire may necessarily be compromised in one or more areas to enable adequate electrosurgical performance.

To minimize wire exchanges and, therefore, maximize procedural efficiency, electrosurgical guidewires may be required to support over-the-wire delivery of bulky devices. Successful delivery of bulky devices may require excellent mechanical performance. For various reasons such as these, a continuing need exists for electrosurgical guidewires having excellent mechanical as well as electrosurgical performance. In other words, it would be beneficial to provide electrosurgical guidewires and related systems and methods with better optimization of mechanical and electrosurgical performance characteristics.

### SUMMARY

In one illustrative embodiment, an electrosurgical guidewire is provided and includes a core wire including a distal core wire portion located at a distal portion of the electrosurgical guidewire, a shaft portion located at a proximal portion of the electrosurgical guidewire, and an active electrode. The shaft portion includes an electrical connection portion configured to provide an electrical connection to an electrosurgical generator. A first electrical insulation material with a first thickness covers the distal core wire portion. A second electrical insulation material with a second thickness covers the shaft portion. The first and second electrical insulation materials may be formed from the same material or from different materials. The first thickness is greater than the second thickness. There may be any desired number of different electrical insulation thickness portions provided along the core wire length, and configurations of the varying thicknesses may include a tapered thickness and/or stepped portions of varying thicknesses.

Various additional and/or optional features of the guidewire may be provided. Some examples are given as follows. The active electrode may be located at a distal end of the distal core wire portion. The distal core wire portion may include a non-uniform surface which may help secure the first electrical insulation material to the distal core wire portion. The non-uniform surface may include physical features, such as bumps, ridges, texturing, or other surface features which may help to secure the first electrical insulation material to the distal core wire portion. The electrosurgical guidewire may include a transition portion extending between the distal core wire portion and the shaft portion. The transition portion may be covered by at least one of the first electrical insulation material or the second electrical insulation material. The transition portion may include an adhesive material and/or a machined portion. The core wire may be stainless steel. At least one of the first electrical insulation material or the second electrical insulation material may be at least one of polytetrafluoroethylene (PTFE), polyethylene terepthalate (PET), fluorinated ethylene propylene (FEP), polyether block amide (PEBA), or lubricious hydrophobic polymeric coating. The first electrical insulation material may be a heat shrink tubing. The second electrical insulation material may be a spray coating. In some embodiments, a portion of the distal core wire portion may be covered by the second electrical insulation material. A portion of the shaft portion may be covered by the first electrical insulation material. The first insulation material may include a tapered thickness. The first insulation material may include portions of different, stepped thicknesses.

An electrosurgical system may include the electrosurgical guidewire and an apparatus for coupling the electrosurgical guidewire to an electrosurgical generator. The apparatus may include an elongated flexible conductive element, an activator unit for selectively controlling energy to the electrosurgical guidewire, and a coupler for removably coupling the electrosurgical guidewire to the apparatus. The electrosurgical system may include an electrosurgical generator.

In another illustrative embodiment or aspect, a method of performing an electrosurgical procedure is provided. The method generally includes directing an electrosurgical guidewire into a patient. The electrosurgical guidewire includes a core wire having a distal core wire portion located at a distal portion of the electrosurgical guidewire. The electrosurgical guidewire includes a shaft portion located at a proximal portion of the electrosurgical guidewire and having an electrical connection portion configured to provide an electrical connection to an electrosurgical generator. The electrosurgical guidewire also includes an active electrode. A first electrical insulation material with a first thickness covers the distal core wire portion. A second electrical insulation material with a second thickness covers the shaft portion. The first thickness is greater than the second thickness. The method further includes vaporizing tissue within the patient using the active electrode.

In alternative embodiments of the method, the active electrode may be located at a distal end of the distal core wire portion. The distal core wire portion may include a non-uniform surface which may help secure the first electrical insulation material to the distal core wire portion. The non-uniform surface may include physical features, such as bumps, ridges, texturing, or other surface features which may help to secure the first electrical insulation material to the distal core wire portion. The electrosurgical guidewire wire may include a transition portion extending between the distal core wire portion and the shaft portion. The transition portion may be covered by at least one of the first electrical insulation material or the second electrical insulation material. The transition portion may include an adhesive material and/or a machined portion. The core wire may be stainless steel. At least one of the first electrical insulation material or the second electrical insulation material may be at least one of PTFE, PET, FEP, PEBA, or lubricious hydrophobic polymeric coating. The first electrical insulation material may be a heat shrink tubing. The second electrical insulation material may be a spray coating. In some embodiments, a portion of the distal core wire portion may be covered by the second electrical insulation material. A portion of the shaft portion may be covered by the first electrical insulation material. The first insulation material may include a tapered thickness. The first insulation material may include portions of different, stepped thicknesses.

In some embodiments of the method, the electrosurgical guidewire may be part of an electrosurgical system. The electrosurgical system may include the electrosurgical guidewire and an apparatus for coupling the electrosurgical guidewire to an electrosurgical generator. The apparatus may include an elongated flexible conductive element, an activator unit for selectively controlling energy to the electrosurgical guidewire, and a coupler for removably coupling the electrosurgical guidewire to the apparatus. The electrosurgical system may also include an electrosurgical generator.

The systems, devices and methods of the invention may further include one or more aspects as discussed herein, as well as even additional aspects. It will be further understood that electrosurgical systems including other components will benefit from aspects of the present invention and may be provided as well.

### BRIEF DESCRIPTION OF THE DRAWINGS

FIG. 1 shows an illustrative electrosurgical guidewire.

FIG. 1A is a perspective view of an illustrative system for delivering RF energy to tissue during a medical procedure including the electrosurgical guidewire of FIG. 1.

FIG. 2 is a cross sectional view taken along line 2-2 of FIG. 1.

FIG. 3 is a cross sectional view taken along line 3-3 of FIG. 1.

FIG. 4 is a cross sectional view taken along the longitudinal central axis of the electrosurgical guidewire of FIG. 1 and showing a transition portion of the electrosurgical guidewire.

FIG. 5 is a cross sectional view taken along the longitudinal central axis of an alternative illustrative electrosurgical guidewire at the distal core wire portion.

### DETAILED DESCRIPTION OF ILLUSTRATIVE EMBODIMENTS

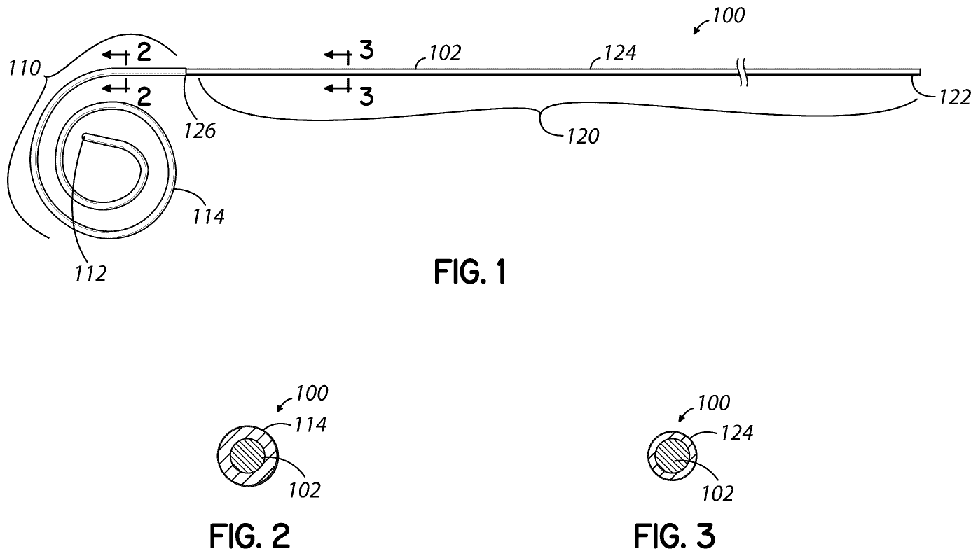

FIG. 1 shows an illustrative electrosurgical guidewire 100. In various embodiments, an electrosurgical guidewire 100 is provided and includes a core wire 102 with a distal core wire portion 110 and a shaft portion 120. The distal core wire portion 110 includes a bare or exposed distal tip 112 or terminus of the electrosurgical guidewire 100 serving as the “active electrode” in the system, where electric current density is highly concentrated to effect tissue vaporization. The core wire 102 may be formed completely or partially of one or more electrically conductive metals. In one embodiment, the core wire 102 is formed of stainless steel. The distal tip 112 of the core wire 102 is bare stainless steel in this embodiment. The electrosurgical guidewire 100 depicted in FIG. 1 includes a pigtail shaped distal core wire portion 110, but a J-tipped, straight-tipped, or other design configuration may be used. The shaft portion 120 has a bare or exposed portion 122 for purposes of electrical connection to, for example, an electrosurgical generator unit supplying radiofrequency (RF) energy. The electrosurgical guidewire 100 together with an electrosurgical generator unit, along with any other desired components useful during medical procedures involving the use of the electrosurgical guidewire 100 and electrosurgical generator unit, can comprise a system in accordance with various embodiments. In some embodiments, these bare or exposed portions 112, 122 of the core wire 102 may be located at respective distal and proximal terminus points or ends. However, in other embodiments, for example, one or both of these bare or exposed portions 112, 122 may be located near but not at a terminus point. In some embodiments, a bare or exposed portion, serving as an active electrode, may be located remotely from the terminus points or ends. The remainder of the electrosurgical guidewire 100 must be insulated to minimize charge dispersion and related loss of electrosurgical effect.

FIG. 1A is a perspective view of an illustrative system for delivering RF energy to tissue during a medical procedure. Further illustrative details of this exemplary system may be found in U.S. patent application Ser. No. 18/243,927, filed on Sep. 8, 2023, the disclosure of which is hereby incorporated by reference herein. In this illustrative embodiment, the electrosurgical guidewire 100, an electrosurgical unit 200, and an apparatus 202 including an elongated flexible conductive element 204, an activator unit 206, and a coupler 208 form the system 210 which is configured for performing a medical procedure. The elongated flexible conductive element 204 is an electrically insulated conductor, such as a wire, configured for transmitting electricity or RF energy. In some embodiments, the elongated flexible conductive element 204 may be a cable including an insulated wire or wires and having a protective casing. The elongated flexible conductive element 204 includes a proximal end 212 including an electrosurgical unit connector 214, and a distal end 216 coupled to the coupler 208. In this illustrative embodiment, the coupler 208 is configured to removably couple the electrosurgical guidewire 100 to the elongated flexible conductive element 204. The electrosurgical unit connector 214, capable of attaching to conventional RF energy generating units for delivering RF energy, may releasably connect to the electrosurgical unit 200. Many commercially available electrosurgical units include a standardized receptacle, such as a monopolar accessory receptacle. The electrosurgical unit connector 214 may be configured to couple to any one of a plurality of electrosurgical units with standardized receptacles. Therefore, the subsystem or assembly comprising, for example, the electrosurgical guidewire 100, and the apparatus 202 including the elongated flexible conductive element 204, activator unit 206, and coupler 208 may be physically coupled to one of several different standardized receptacles of an electrosurgical RF generating unit. In the illustrative embodiment the elongated flexible conductive element 204 has a mid-portion 218 that is connected to the activator unit 206. The activator unit 206 is situated at a location spatially separated from the proximal end 212 of the elongated flexible conductive element 204 and, therefore also spatially separated from the electrosurgical unit 200, by a segment of the elongated flexible conductive element 204. The activator unit 206 also may be spatially separated from the coupler 208 by another segment of the elongated flexible element 204, as shown in FIG. 1A or, optionally, the activator unit 206 may be integrated with or otherwise fixed to the coupler 208. In some embodiments, the activator unit 206 may be located at the proximal end 212 of the elongated flexible conductive element 204 adjacent to or otherwise fixed to the electrosurgical unit connector 214. The activator unit 206 includes a switch element 220, such as a push button or other element, allowing the user to selectively activate the flow of RF energy. The apparatus 202 includes an electroanatomical mapping connector 222 that is capable of connecting to electroanatomical mapping systems.

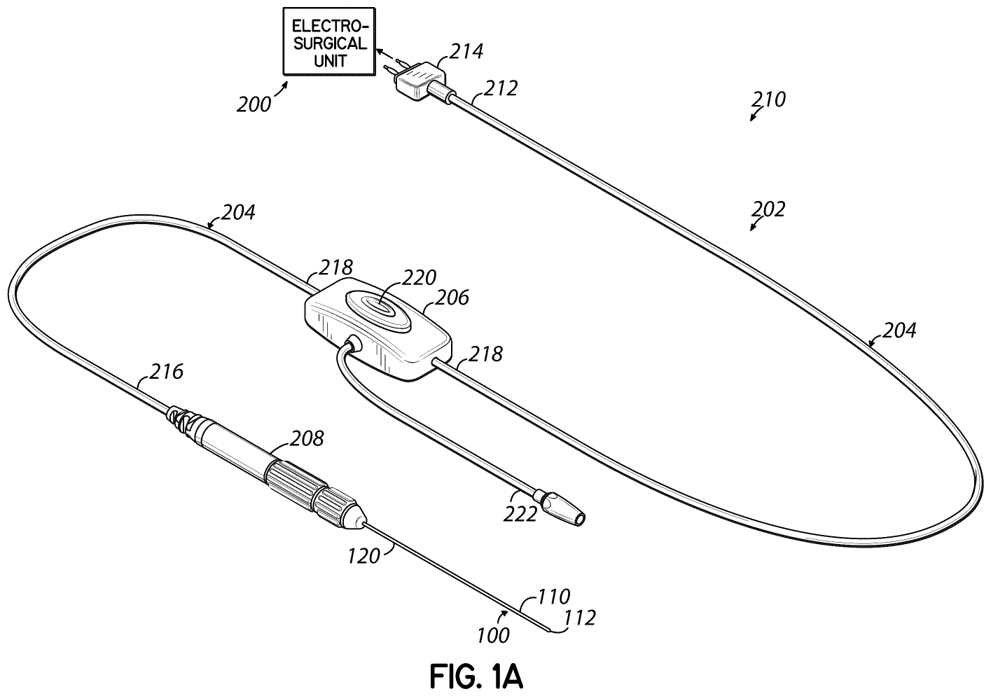

The core wire 102 of the electrosurgical guidewire 100 is variably insulated and includes a first electrical insulation material 114 with a first thickness covering the distal core wire portion 110 and a second electrical insulation material 124 with a second thickness covering the shaft portion 120. FIGS. 2 and 3 show cross sections of the electrosurgical guidewire 100 and illustrate the greater first thickness of the first electrical insulation material 114 and the lesser second thickness of the second electrical insulation material 124. The thicker first electrical insulation material 114 is needed near the bare or exposed distal tip 112, to prevent charge dispersion. Charge dispersion may render the electrosurgical guidewire's active electrode electrosurgically ineffective, especially as it is advanced from within a necessary and inherently insulative catheter, such as a transseptal introducer set comprising a sheath and dilator. In the case of a transseptal introducer set's tip not being in direct contact with the inter-atrial septum, inadequate insulation near the core wire's bare or exposed distal tip 112 may cause the electrosurgical guidewire 100 to fail to puncture the inter-atrial septum as it is advanced from within the transseptal introducer set. By extending adequate insulation a distance more proximally from the core wire's bare or exposed distal tip 112, a longer length of electrosurgically effective guidewire may be established to enable successful inter-atrial septal puncture even in anatomically challenging cases where the transseptal introducer set's tip cannot be brought into direct contact with or even in close proximity to the inter-atrial septum.

The variably insulated core wire 102 includes a lesser amount of electrical insulation material over the remainder of its length, where the need for insulation is not as great. In contrast to the distal core wire portion 110, especially near the bare or exposed distal tip 112, the core wire 102 needs less electrical insulation material along the remainder of its length because the necessary catheter that surrounds the electrosurgical guidewire 100 is inherently insulative in nature. In this regard, the “remainder” of the length may be that portion which is contained within the surrounding catheter and extending proximally, typically onto a sterile field. A “remainder” of the length may be less than the entire length in some embodiments. In this illustrative embodiment, the shaft portion 120 is covered by a second electrical insulation material 124 with a second thickness that is less than the first thickness of the first electrical insulation material 114.

In this illustrative embodiment, the electrosurgical guidewire 100 has a maximum outer diameter of 0.032 inches, a limitation imposed to facilitate compatibility with introducer sets commonly used in inter-atrial septal puncture. The distal core wire portion 110, including the portion near the distal tip 112, may be covered with a first electrical insulation material 114, such as PTFE, PET, FEP, PEBA (such as Pebax®), or alternative heat shrink tubing having a recovered wall thickness of approximately 0.002 to 0.006 inches. If the distal core wire portion 110 has a smooth surface contour, there may be little resistance to undesirable sliding of the first electrical insulation material either proximally or distally along the core wire 102 and its distal portion 110. To help secure the first electrical insulation material 114 to the distal core wire portion 110, the distal core wire portion 110 may include a non-uniform surface. The non-uniform surface may include physical features, such as bumps, ridges, texturing, or other surface features. The remainder (or most of the remainder) of the electrosurgical guidewire 100 lying proximal to the distal core wire portion 110, such as the shaft portion 120, may be insulated with spray coating having a thickness of roughly 0.0001 to 0.002 inches. The spray coating may be PTFE, a lubricious hydrophobic polymeric coating, or any low- or no-PTFE alternative, such as GlideMed®, for example.

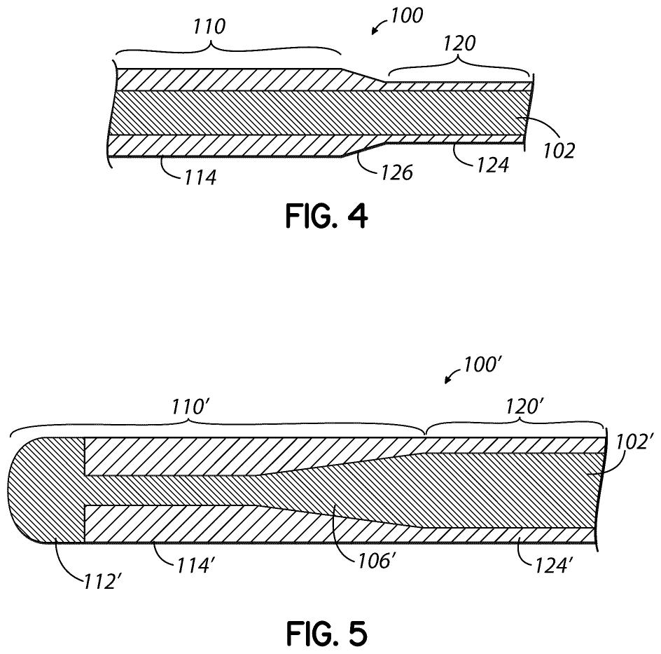

The electrosurgical guidewire 100 also includes a transition portion 126, as shown in greater detail in FIG. 4, that transitions from the second insulation material 124 to the first electrical insulation material 114. The transition portion 126 may be insulated by the first electrical insulation material 114 and/or the second insulation material 124. The different thicknesses of the electrical insulation materials 114, 124 of the electrosurgical guidewire 100 allow mechanical and electrosurgical performance to be optimized in a single guidewire, improving procedural efficiency by enabling over-the-wire delivery of bulky devices immediately after tissue vaporization. The transition portion 126 facilitates smooth passage of catheters and other devices over the different thicknesses of the electrical insulation materials 114, 124. In this case the ramped or tapered transition portion 126 is created with adhesive, but the transition portion 126 may be created with a separate machined part affixed to or otherwise part of the core wire 102 or any other suitable method.

FIG. 5 is a partial section view of an alternative illustrative electrosurgical guidewire 100′. Generally, the electrosurgical guidewire 100′ is similar in construction and operation to the electrosurgical guidewire 100 described above, and the electrosurgical guidewire 100′ may be substituted for other electrosurgical guidewires, or any electrosurgical guidewire 100′ may be used, in various other exemplary embodiments according to the present disclosure. Like reference numbers refer to like components. For brevity, the following description minimizes redundant description and focuses on the differences between the electrosurgical guidewire 100 and the electrosurgical guidewire 100′.

In the setting of a constrained outer guidewire diameter (such as to less than 0.032 inches to facilitate compatibility with necessary catheters), the outer diameter of the core wire 102′ may be smaller near the bare or exposed distal tip 112′ (where more insulation is needed) and may be larger over the remainder of its length, such as the shaft portion 120′ for example, where less insulation is needed. A core wire transition portion 106′ allows for a gradual transition from the larger diameter of the shaft portion 120′ to the smaller diameter of the distal core wire portion 110′. Thus, mechanical performance may be prioritized over the shaft portion 120′ of the electrosurgical guidewire 100′, which must be stiff enough to support over-the-wire delivery of bulky devices. Conversely, electrosurgical performance is prioritized near the bare or exposed distal tip 112′, where mechanical performance requirements do not have priority. In this illustrative example, the shaft portion 120′ of the core wire 102′ is roughly 0.031 inches in diameter, and the distal core wire portion 110′ of the core wire 102′ is tapered to a smaller diameter that allows the maximum outer diameter of the electrosurgical guidewire 100′ to remain less than 0.032 inches after application of the PTFE heat shrink tubing. Here, the thickness of the first and second electrical insulation materials 114′, 124′ vary in more than two thicknesses over a length of the core wire 102′. The thickness may, for example, vary in a tapered manner and/or in a more stepped manner of two or more different thicknesses. The distal core wire portion 110′ may include a non-uniform surface which may help secure the first electrical insulation material 114′ to the distal core wire portion 110′. The non-uniform surface may include physical features, such as bumps, ridges, texturing, or other surface features. One of many possible alternatives is shown in cross section with the electrical insulation materials 114′, 124′ illustrated in FIG. 5. This type of design may provide advantages, such as: 1) eliminating one or more transition point(s) on the electrosurgical guidewire 100′ having variable thickness of electrical insulation, 2) improving the ease of catheter passage over the core wire 102′, 3) reducing risk of peeling or embolization of the insulation materials 114′, 124′ during a medical procedure, and 4) simplifying manufacture of the electrosurgical guidewire 100′.

While the present invention has been illustrated by the description of specific embodiments thereof, and while the embodiments have been described in considerable detail, it is not intended to restrict or in any way limit the scope of the appended claims to such detail. The various features discussed herein may be used alone or in any combination within and between the various embodiments. Additional advantages and modifications will readily appear to those skilled in the art. The invention in its broader aspects is therefore not limited to the specific details, representative devices, systems and methods and illustrative examples shown and described. Accordingly, departures may be made from such details without departing from the scope or spirit of the general inventive concept.

What is claimed is:

1. An electrosurgical guidewire, comprising:
a core wire including:
a distal core wire portion located at a distal portion of the electrosurgical guidewire,
a shaft portion located at a proximal portion of the electrosurgical guidewire and including an electrical connection portion configured to provide an electrical connection to an electrosurgical generator, and
an active electrode;
a first electrical insulation material with a first thickness covering the distal core wire portion; and
a second electrical insulation material with a second thickness covering the shaft portion, wherein the first thickness is greater than the second thickness.

2. The electrosurgical guidewire of claim 1, wherein the active electrode is located at a distal end of the distal core wire portion.

3. The electrosurgical guidewire of claim 1, wherein the distal core wire portion further comprises a non-uniform surface for securing the first electrical insulation material to the distal core wire portion.

4. The electrosurgical guidewire of claim 1, further comprising a transition portion, extending between the distal core wire portion and the shaft portion, and covered by at least one of the first electrical insulation material or the second electrical insulation material.

5. The electrosurgical guidewire of claim 4, wherein the transition portion comprises an adhesive material.

6. The electrosurgical guidewire of claim 4, wherein the transition portion comprises a machined portion.

7. The electrosurgical guidewire of claim 1, wherein the core wire comprises stainless steel.

8. The electrosurgical guidewire of claim 1, wherein at least one of the first electrical insulation material or the second electrical insulation material comprises at least one of PTFE, PET, FEP, PEBA, or lubricious hydrophobic polymeric coating.

9. The electrosurgical guidewire of claim 1, wherein the first electrical insulation material comprises a heat shrink tubing.

10. The electrosurgical guidewire of claim 1, wherein the second electrical insulation material comprises a spray coating.

11. The electrosurgical guidewire of claim 1, wherein a portion of the distal core wire portion is covered by the second electrical insulation material.

12. The electrosurgical guidewire of claim 1, wherein a portion of the shaft portion is covered by the first electrical insulation material.

13. The electrosurgical guidewire of claim 1, wherein the first insulation material comprises a tapered thickness.

14. The electrosurgical guidewire of claim 1, wherein the first insulation material comprises portions of different, stepped thicknesses.

15. An electrosurgical system comprising:
the electrosurgical guidewire of claim 1; and
an apparatus for coupling the electrosurgical guidewire to an electrosurgical generator, the apparatus including:
an elongated flexible conductive element,
an activator unit for selectively controlling energy to the electrosurgical guidewire, and
a coupler for removably coupling the electrosurgical guidewire to the apparatus.

16. The electrosurgical system of claim 15, further comprising an electrosurgical generator.

17. A method of performing an electrosurgical procedure, comprising:
directing an electrosurgical guidewire into a patient, the electrosurgical guidewire including a core wire having a distal core wire portion located at a distal portion of the electrosurgical guidewire, a shaft portion located at a proximal portion of the electrosurgical guidewire and including an electrical connection portion configured to provide an electrical connection to an electrosurgical generator, an active electrode, a first electrical insulation material with a first thickness covering the distal core wire portion, a second electrical insulation material with a second thickness covering the shaft portion, the first thickness being greater than the second thickness; and
vaporizing tissue within the patient using the active electrode.

18. The method of claim 17, wherein the active electrode is located at a distal end of the distal core wire portion.

19. The method of claim 17, wherein the distal core wire portion further comprises a non-uniform surface for securing the first electrical insulation material to the distal core wire portion.

20. The method of claim 17, wherein the electrosurgical guidewire further comprises a transition portion, extending between the distal core wire portion and the shaft portion, and covered by at least one of the first electrical insulation material or the second electrical insulation material.

21. The method of claim 20, wherein the transition portion further comprises an adhesive material.

22. The method of claim 20, wherein the transition portion further comprises a machined portion.

23. The method of claim 17, wherein the core wire comprises stainless steel.

24. The method of claim 17, wherein at least one of the first electrical insulation material or the second electrical insulation material comprises at least one of PTFE, PET, FEP, PEBA, or lubricious hydrophobic polymeric coating.

25. The method of claim 17, wherein the first electrical insulation material comprises a heat shrink tubing.

26. The method of claim 17, wherein the second electrical insulation material comprises a spray coating.

27. The method of claim 17, wherein a portion of the distal core wire portion is covered by the second electrical insulation material.

28. The method of claim 17, wherein a portion of the shaft portion is covered by the first electrical insulation material.

29. The method of claim 17, wherein the first insulation material comprises a tapered thickness.

30. The method of claim 17, wherein the first insulation material comprises portions of different, stepped thicknesses.

31. The method of claim 17, wherein the electrosurgical guidewire is part of an electrosurgical system comprising the electrosurgical guidewire and an apparatus for coupling the electrosurgical guidewire to an electrosurgical generator, the apparatus includes an elongated flexible conductive element, an activator unit for selectively controlling energy to the electrosurgical guidewire, and a coupler for removably coupling the electrosurgical guidewire to the apparatus.

32. The method of claim 31, wherein the electrosurgical system further comprises an electrosurgical generator.
